(19) United States
(12) Patent Application Publication
(10) Pub. No.: US 2025/0263457 A1
(43) Pub. Date: Aug. 21, 2025

(54) Pharmaceutical Composition for Preventing and Treating Liver Disease Comprising Novel Peptide

(71) Applicant: Pusan National University Industry-University Cooperation Foundation, Busan (KR)

(72) Inventors: Youngmi Jung, Busan (KR); Jinsol Han, Busan (KR); Chanbin Lee, Busan (KR)

(21) Appl. No.: 19/058,441

(22) Filed: Feb. 20, 2025

(30) Foreign Application Priority Data
Feb. 21, 2024 (KR) 10-2024-0024863

(51) Int. Cl. C07K 14/525; A61K 38/00; A61P 1/16

(52) U.S. Cl. CPC C07K 14/525; A61P 1/16; A61K 38/00

(57) ABSTRACT

The present disclosure relates to a TSG-6 fragment and a pharmaceutical composition for preventing or treating liver disease including the TSG-6 fragment as an active ingredient. The TSG-6 fragment according to the present disclosure inhibited the cleavage of CD44 by MMP-14 in human hepatic stellate cells, thereby inhibiting the expression of genes related to hepatic stellate cell activation and fibrosis. In addition, the TSG-6 fragment alleviated liver damage and inflammation in an alcohol-related liver disease mouse model and inhibited fatty liver and liver fibrosis. Therefore, the TSG-6 fragment according to the present disclosure may be used as various therapeutic agents for liver diseases accompanied by liver damage and liver fibrosis.

### CROSS-REFERENCE TO RELATED APPLICATION

This present application claims the benefit of priority to Korean Patent Application No. 10-2024-0024863, entitled “PHARMACEUTICAL COMPOSITION FOR PREVENTING AND TREATING LIVER DISEASE COMPRISING NOVEL PEPTIDE” filed on Feb. 21, 2024, in the Korean Intellectual Property Office, the entire disclosure of which is incorporated herein by reference.

### REFERENCE TO AN ELECTRONIC SEQUENCE LISTING

The contents of the electronic sequence listing (KAIP-003_SEQ_LIST.xml; Size: 12,243 bytes; and Date of Creation: Feb. 20, 2025) is herein incorporated by reference in its entirety.

### FIELD

The present disclosure relates to a tumor necrosis factor-inducible gene 6 protein (TSG-6) fragment and a pharmaceutical composition for preventing or treating liver disease including the TSG-6 fragment as an active ingredient.

apeutic agent that may be safely taken and administered long-term is urgently needed.

### BACKGROUND

Liver disease can be induced by various factors, including excessive alcohol consumption, exposure to toxic substances and heavy metals, and a high-fat diet. Chronic progression of liver diseases, such as alcohol-related liver disease, metabolic dysfunction-associated steatotic liver disease, and hepatitis, may ultimately lead to cirrhosis or hepatocellular carcinoma, inducing significant health risks.

Liver disease is a serious health problem worldwide, causing more than 2 million deaths worldwide each year, accounting for approximately 4% of all deaths worldwide according to the World Health Organization (WHO) database (Devarbhavi et al., J Hepatol. 79:516-537 (2023)). Despite the growing prevalence of liver disease and its significant disease burden, there is currently no effective and safe therapeutic agent for liver disease, including alcohol-related liver disease, metabolic dysfunction-associated steatotic liver disease, and liver fibrosis. Recently, a chemically modified drug of ibipinapant, a type of brain-penetrating cannabinoid type 1 (CB-1) receptor antagonist, has been reported, but its actual efficacy as a drug is unknown (Cinar et al., JCI Insight. 1: e87336 (2016)). Therefore, the development of a novel liver disease the

### SUMMARY

Accordingly, the present inventors conducted research to develop a safe long-term therapeutic agent for liver disease, and as a result, completed the present disclosure by confirming that a fragment of TSG-6 inhibited liver fibrosis.

In order to achieve the object, one aspect of the present disclosure provides a tumor necrosis factor-inducible gene 6 protein (TSG-6) fragment and a pharmaceutical composition for preventing or treating liver disease including the TSG-6 fragment as an active ingredient.

Another aspect of the present disclosure provides a polynucleotide encoding the TSG-6 fragment.

Yet another aspect of the present disclosure provides a method for preventing or treating liver disease, including administering the TSG-6 fragment or the pharmaceutical composition including the TSG-6 fragment.

According to the present disclosure, the TSG-6 fragment inhibited the cleavage of cluster of differentiation 44 (CD44) by matrix metallopeptidase 14 (MMP-14) in human hepatic stellate cells (HSCs), thereby inhibiting the activation of HSCs and the expression of fibrosis markers. In addition, the TSG-6 fragment alleviated liver damage and inflammation in an alcohol-related liver disease mouse model, and inhibited fatty liver and liver fibrosis. Therefore, the TSG-6 fragment according to the present disclosure may be used as various therapeutic agents for liver diseases accompanied by liver damage and liver fibrosis.

### BRIEF DESCRIPTION OF THE DRAWINGS

The above and other aspects, features, and advantages of the present disclosure will become apparent from the detailed description of the following aspects in conjunction with the accompanying drawings, in which:

FIG. 1A is a diagram illustrating amino acid sequences and structures of TSG-6 mimetic candidate peptides;

FIG. 1B is a diagram illustrating a result of analyzing the binding affinity of the candidate peptides of FIG. 1A with MMP-14 and CD44 using HADDOCK2.4;

FIG. 1C is a diagram illustrating a result of predicting a structure of a binding complex of peptide 4 (hereinafter, peptide YJ) of FIG. 1A and MMP-14 and CD44;

FIG. 2A is a diagram illustrating a schematic diagram of an experimental method for confirming a liver fibrosis inhibition effect of peptide YJ in human primary hepatic stellate cells (pHSCs);

FIG. 2B is a graph showing a result of measuring cell proliferation after treating human pHSCs with peptide YJ for each concentration of 10, 20, 40, and 100 ng/ml for 24 or 48 hours. Mean±SEM, one-way analysis of variance (ANOVA) followed by post hoc Tukey's test;

FIG. 2C is a diagram illustrating a result of confirming the expression of HSC activation and HSC deactivation marker proteins through Western blot, after treating human pHSCs with peptide YJ for each concentration of 20 and 40 ng/ml for 24 or 48 hours;

FIG. 3A is a diagram illustrating a result of confirming the expression of a CD44 intracellular domain (CD44ICD) in nuclear proteins through Western blot, after treating human pHSCs with peptide YJ (40 ng/ml) for 0, 6, 12, or 24 hours (top), and a graph showing a result of quantitative analysis thereof (bottom). Mean±SEM, one-way ANOVA followed by post hoc Tukey's test, *p<0.05 vs vehicle;

FIG. 3B is a diagram illustrating a result of confirming the expression of CD44ICD (red) and α-SMA (α-smooth muscle actin, green) using immunofluorescence, after treating human pHSCs with peptide YJ (40 ng/ml) for 6, 12, or 24 hours. DAPI: nuclear staining;

FIG. 3C is a graph showing a result of confirming the expression of mRNA of a fibrosis marker protein through RT-qPCR, after treating human pHSCs with peptide YJ (40 ng/ml) for 24 or 48 hours. n=3, Mean±SEM, one-way ANOVA followed by post hoc Tukey's test, *p<0.05, **p<0.005 vs vehicle for 24 hours, #p<0.05 vs vehicle for 48 hours;

FIG. 3D is a diagram illustrating a result of confirming the expression of CD44, CD44ICD, and HSC activation/deactivation marker proteins through Western blot, after treating human pHSCs with peptide YJ (40 ng/ml) for 24 or 48 hours;

FIG. 4A is a diagram illustrating an experimental schedule for establishing an alcohol-related liver disease mouse model and confirming a liver disease treatment effect of peptide YJ using the alcohol-related liver disease mouse model;

FIG. 4B is a diagram illustrating a result of comparing liver tissues of mice which were exposed to control (Pair) or ethanol-containing (EtOH) diet with or without (non-treated) vehicle;

FIG. 4C is a diagram illustrating a result of comparing liver tissue weights (relative weight to body weight) of mice which were exposed to control (Pair) or ethanol-containing (EtOH) diet with or without (non-treated) vehicle. Mean±SEM, one-way ANOVA followed by post hoc Tukey's test, *p<0.05;

FIG. 4D is a graph showing a result of comparing blood alanine aminotransferase (ALT) and aspartate aminotransferase (AST) levels in serum of mice which were exposed to control (Pair) or ethanol-containing (EtOH) diet with or without (non-treated) vehicle. Mean±SEM;

FIG. 4E is a diagram showing a result of comparing triglyceride (TG) levels in liver tissue of mice which were exposed to control (Pair) or ethanol-containing (EtOH) diet with or without (non-treated) vehicle. Mean±SEM, one-way ANOVA followed by post hoc Tukey's test, **p<0.005;

FIG. 4F is a diagram illustrating a result of comparing the liver tissue morphology, triglycerides, and the expression of CD68 and F4/80 proteins in the liver tissue of mice which were exposed to control (Pair) or ethanol-containing (EtOH) diet with or without (non-treated) vehicle through hematoxylin and eosin (H&E), Oil red O staining, and immunohistochemistry (IHC). Scale bar=50 μm, H&E; hematoxylin and eosin staining, red: Oil red O;

FIG. 5A is a graph showing a result of comparing the mRNA expression of fibrosis marker in the liver tissue of mice which were exposed to control (Pair) or ethanol-containing (EtOH) diet with or without (non-treated) vehicle through RT-qPCR. n=3, Mean±SEM, one-way ANOVA followed by post hoc Tukey's test, *p<0.05 vs pair;

FIG. 5B is a diagram illustrating a result of comparing the expression of fibrosis marker proteins of mice which were exposed to control (Pair) or ethanol-containing (EtOH) diet with or without (non-treated) vehicle through Western blot;

FIG. 5C is a graph showing a result of measuring and comparing collagen in liver tissue of mice which were exposed to control (Pair) or ethanol-containing (EtOH) diet with or without (non-treated) vehicle through Hydroxyproline assay. Mean±SEM, one-way ANOVA followed by post hoc Tukey's test, *p<0.05;

FIG. 5D is a diagram illustrating a result of comparing the expression of collagen and α-SMA in the liver tissue of mice which were exposed to control (Pair) or ethanol-containing (EtOH) diet with or without (non-treated) vehicle through Sirius red staining and IHC, respectively. Scale bar=50 μm;

FIG. 6A is a diagram illustrating a result of confirming the size and shape of liver tissue of vehicle or peptide YJ-treated mice fed pair or EtOH diet;

FIG. 6B is a graph showing relative liver weight to body weight ratio of vehicle or peptide YJ-treated mice fed pair or EtOH diet. Mean±SEM, one-way ANOVA followed by post hoc Tukey's test, *p<0.05;

FIG. 6C is a graph showing a result of confirming AST and ALT in serum of vehicle or peptide YJ-treated mice fed pair or EtOH diet. Mean±SEM, one-way ANOVA followed by post hoc Tukey's test, *p<0.05;

FIG. 6D is a graph showing a result of measuring triglycerides in liver tissue of vehicle or peptide YJ-treated mice fed pair or EtOH diet. Mean±SEM, one-way ANOVA followed by post hoc Tukey's test, **p<0.005;

FIG. 6E is a diagram illustrating a result of confirming the morphology of liver tissue and the expression of CD68 (cluster of differentiation 68) and F4/80 proteins through H&E staining and immunohistochemical staining, respectively, in livers from vehicle or peptide YJ-treated mice fed pair or EtOH diet. Scale bar=50 μm, H&E: hematoxylin and eosin staining;

FIG. 6F is a diagram illustrating a result of confirming the expression of alcohol metabolism-related proteins (cytochrome P450 2E1 (CYP2E1) and 4-hydroxy-2-noneal (4-HNE)) and apoptosis-related proteins (caspase-3 and cleaved caspase-3) through Western blot, in livers from vehicle or peptide YJ-treated mice fed pair or EtOH diet;

FIG. 7A is a graph showing a result of confirming mRNA expression levels of fibrosis marker (α-SMA, vimentin, and COL1α1) in liver tissue from vehicle or peptide YJ-treated mice fed pair or EtOH diet through RT-qPCR. n=3, Mean±SEM, one-way ANOVA followed by post hoc Tukey's test, *p<0.05, **p<0.005;

FIG. 7B is a diagram illustrating a result of confirming the expression of fibrosis marker proteins in liver tissue from vehicle or peptide YJ-treated mice fed pair or EtOH diet through Western blot;

FIG. 7C is a graph showing a result of confirming the concentration of collagen in liver tissue from vehicle or peptide YJ-treated mice fed pair or EtOH diet through Hydroxy proline assay. Mean±SEM, one-way ANOVA followed by post hoc Tukey's test, **p<0.005;

FIG. 7D is a diagram illustrating a result of confirming the expression of collagen and α-SMA in liver tissue from vehicle or peptide YJ-treated mice fed pair or EtOH diet through Sirius staining and immunohistochemical staining;

FIG. 8A is a diagram illustrating a result of confirming the expression of CD44 in liver tissue from vehicle or peptide YJ-treated mice fed pair or EtOH diet through immunohistochemical staining. Scale bar=50 μm;

FIG. 8B is a diagram illustrating a result of confirming the expression of CD44 and CD44ICD in whole lysate of liver tissue from vehicle or peptide YJ-treated mice fed pair or EtOH diet through Western blot; and

FIG. 8C is a diagram illustrating a result of confirming the expression of CD44 and CD44ICD in nuclear protein of liver tissue from vehicle or peptide YJ-treated mice fed pair or EtOH diet through Western blot, and a graph showing quantitative analysis thereof. Mean±SEM, one-way ANOVA followed by post hoc Tukey's test. *p<0.05 vs vehicle.

### DETAILED DESCRIPTION

### TSG-6 Fragment

One aspect of the present disclosure provides a TSG-6 fragment.

As used in the present disclosure, the term “TSG-6 (tumor necrosis factor-inducible gene 6 protein)” refers to a 30 kDa secretory protein containing a hyaluronan-binding LINK domain. The TSG-6 reconstructs an extracellular matrix (ECM) through the hyaluronan-binding domain and is involved in the stability of the extracellular matrix and cell migration. In addition, the TSG-6 is known to exhibit an anti-inflammatory effect in various organs. In a recent study, it has been reported that TSG-6 increases autophagy influx in hepatocytes of chronically damaged liver and promotes liver regeneration by reducing fibrosis and inflammation.

In the present disclosure, the TSG-6 fragment may be a polypeptide represented by the following Structural Formula I:

[Core Domain]−[C-Terminal Extension Domain] (I)

In Structural Formula I, the core domain is a polypeptide including an amino acid sequence represented by SEQ ID NO: 1, and the C-terminal extension domain may have 0 to 33 amino acids sequentially deleted from the C-terminal to the N-terminal starting from an amino acid at position 33 of SEQ ID NO: 9.

The core domain refers to a polypeptide having an amino acid sequence at positions 43 to 56 of full-length TSG-6. At this time, the full-length TSG-6 may include an amino acid sequence represented by SEQ ID NO: 10.

The C-terminal extension domain is a domain that is bound to the C-terminal of the core domain, and refers to a polypeptide including an amino acid sequence at positions 57 to 89 of full-length TSG-6. As a specific example, the C-terminal extension domain may include an amino acid sequence represented by SEQ ID NO: 9.

The C-terminal extension domain may be a polypeptide having an amino acid sequence represented by SEQ ID NO: 9, or may have 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or 33 amino acid residues deleted from the C-terminal to the N-terminal starting from the amino acid at position 33 of the polypeptide.

Specifically, the C-terminal extension domain may be a polypeptide having an amino acid sequence represented by SEQ ID NO: 9, or may have 0, 19, 25, or 33 amino acid residues deleted from the C-terminal to the N-terminal starting from the amino acid at position 33 of the polypeptide.

In one example of the present disclosure, the C-terminal extension domain may be a polypeptide including or consisting of any one amino acid sequence selected from the group consisting of SEQ ID NOs: 7 to 9.

In one example of the present disclosure, the TSG-6 fragment, the core domain, and the C-terminal extension domain may include amino acid sequences of Table 1 below. The following amino acid sequences are sequentially described in order from the N-terminal to the C-terminal.

In one example of the present disclosure, the TSG-6 fragment may include any one amino acid sequence selected from the group consisting of SEQ ID NOs: 1 to 4.

TABLE 1

| TSG-6 | Core | C-terminal extension |
|---|---|---|
| fragment | domain | domain (N -> C) |
| Peptide 1 | RSGKYKLT | |
| (SEQ ID NO: 1) | YAEAKA | |
| Peptide 2 | RSGKYKLT | VCEFEGGH |
| (SEQ ID NO: 2) | YAEAKA | (SEQ ID NO: 7) |
| Peptide 3 | RSGKYKLT | VCEFEGGHLATYKQ |
| (SEQ ID NO: 3) | YAEAKA | (SEQ ID NO: 8) |
| Peptide 4 | RSGKYKLT | VCEFEGGHLATYK |
| (SEQ ID NO: 4) | YAEAKA | QLEAARKIGFHVC |
| | | AAGWMAK |
| | | (SEQ ID NO: 9) |

In addition, the TSG-6 fragment may consist of an amino acid sequence having about 80%, about 85%, about 90%, about 95%, about 96%, about 97%, about 98%, about 99% or 100% identity with the amino acid sequences represented by SEQ ID NOs: 1 to 4, respectively, as long as it has the same activity as the TSG-6 fragment. In particular, preferably, the TSG-6 fragment of the present disclosure may consist of an amino acid sequence having 90% or more homology with the amino acid sequence represented by SEQ ID NO: 4. In this case, while including the core domain represented by SEQ ID NO: 1, 1, 2, or 3 substitutions may be made in the amino acid sequence represented by SEQ ID NO: 9, and it is preferable that these amino acid substitutions are conservative substitutions that do not change the properties of the protein.

Here, the “same activity” means a state of exhibiting all or almost the same degree of a specific property. Specifically, the “same activity” means an effect of improving liver diseases such as inhibition of fatty liver and inhibition of liver fibrosis.

In addition, the polypeptide of the present disclosure also includes a polypeptide derivative in which a part of a chemical structure of the polypeptide is modified while maintaining a basic skeleton and activity of the polypeptide according to the present disclosure. For example, the polypeptide of the present disclosure includes structural modifications for changing the stability, storage, volatility, solubility or the like of the polypeptide of the present disclosure. In particular, in the case of a chemically synthesized peptide, since the N and C terminals are charged, the N terminal may be acetylated or/and the C terminal may be amidated in order to remove these charges, but is not particularly limited thereto.

In the present disclosure, the TSG-6 fragment may mutually bind to CD44 and MMP-14 proteins. Specifically, the TSG-6 fragment may mutually bind to a binding complex of the CD44 and MMP-14 proteins to inhibit cleavage of the CD44 protein by the MMP-14 protein. In one example of the present disclosure, the expression of CD44 is increased in activated HSCs that promote liver fibrosis, and the CD44 is cleaved into CD44ICD by MMP-14. The CD44ICD migrates into the nucleus of HSCs and promotes the expression of HSC activation markers. The TSG-6 fragment according to the present disclosure binds to the complex of CD44 and MMP-14, and inhibits the generation and nuclear translocation of CD44ICD, thereby inhibiting the activity of HSCs and thus inhibiting liver fibrosis.

### Polynucleotide

Another aspect of the present disclosure provides a polynucleotide encoding the TSG-6 fragment. At this time, the TSG-6 fragment is the same as described above. Specifically, the polynucleotide may include a base sequence encoding a polypeptide consisting of an amino acid sequence represented by SEQ ID NOs: 1 to 4, respectively.

In addition, if the polynucleotide encodes the same TSG-6 fragment, one or more bases may be mutated by substitution, deletion, insertion, or a combination thereof. When the polynucleotide sequence is chemically synthesized and manufactured, a synthetic method widely known in the art, for example, a method described in the literature, may be used, and examples thereof may include triester, phosphate, phospho amidite, and H-phosphate methods, PCR and other autoprimer methods, oligonucleotide synthesis methods on solid supports, and the like.

Specifically, the polynucleotide may include a nucleic acid sequence having at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% identity to a base sequence encoding the TSG-6 fragment including the amino acid sequences represented by each of SEQ ID NOs: 1 to 4.

### Pharmaceutical Composition and Treatment Method

Another aspect of the present disclosure provides a pharmaceutical composition for preventing or treating liver disease including the TSG-6 fragment as an active ingredient. In addition, the present disclosure provides a method for preventing or treating liver disease, including administering the TSG-6 fragment or the pharmaceutical composition including the TSG-6 fragment to a subject in need thereof.

The liver disease may be selected from the group consisting of fatty liver, hepatitis, liver fibrosis, cirrhosis, liver failure, and liver cancer. In addition, the liver disease may be alcohol-related liver disease.

The fatty liver means that a greater amount of fat is accumulated in the liver than the proportion (about 5%) of fat in a normal liver. The fatty liver may be alcohol-related fatty liver, or metabolic dysfunction-associated steatotic liver disease caused by obesity, diabetes, hyperlipidemia, or drugs.

The hepatitis refers to inflammation of hepatocytes and liver tissue, and if a process of destroying and regenerating hepatocytes over a long period of time due to chronic hepatitis is repeated, fibrous tissue and regenerative nodules are accumulated in the liver, leading to liver cirrhosis. If the liver cirrhosis progresses above a certain level, the liver cirrhosis may cause complications such as hepatic encephalopathy and esophageal varix.

The liver fibrosis is caused by a chronic inflammatory response in the liver tissue, and the causes of this inflammatory response are known to be hepatitis caused by viral infection, alcohol consumption, drug abuse, autoimmune disease, metabolic disorder, cholestasis, and hepatitis caused by other causes. When the liver fibrosis progresses to liver cirrhosis, the liver fibrosis becomes an irreversible chronic disease in which normal restoration of liver tissue is difficult, and accordingly, the liver fibrosis progresses to decompensated liver cirrhosis or liver cancer, and as a result, the mortality rate continues to increase.

The liver failure is a condition in which the synthesis and detoxification functions of the liver are reduced, and occurs when hepatitis and liver cirrhosis coexist.

As used in the present disclosure, the term “prevention” may comprehensively mean preventing a disease in advance or reducing the possibility or frequency of occurrence by administering the pharmaceutical composition in a pharmaceutically effective amount. The “pharmaceutically effective amount” has the same meaning as “therapeutically effective amount” and may be easily determined by those skilled in the art according to factors well-known in the medical field, such as a type of disease, age, weight, health, and sex of a patient, sensitivity to a drug of a patient, a route of administration, a method of administration, a frequency of administration, a duration of treatment, and drugs to be combined or used simultaneously.

As used in the present disclosure, the term “treatment” may comprehensively mean improving a disease by administering the pharmaceutical composition in a pharmaceutically effective amount, and may be providing relief or cure of the symptoms of the disease in a shortened period of time compared to natural healing, and improving one or most of symptoms caused by the disease. The pharmaceutically effective amount is the same as described above. The pharmaceutical composition of the present disclosure may be a composition for treating a disease by itself, or may also be administered together with other pharmacological ingredients and applied as a therapy adjuvant for a disease. Accordingly, the “treatment” includes the meaning of “treatment aid”.

Meanwhile, the pharmaceutical composition of the present disclosure is administered in a “therapeutically effective amount”. The therapeutically effective amount is the same as described above.

As used in the present disclosure, the term “administration” means introducing a predetermined substance into a subject by a suitable method, and the composition may be administered through any general route so long as the composition may reach a target tissue. Preferably, the administration may be parenteral administration, and the parenteral administration may be intraperitoneal administration, intravenous administration, intramuscular administration, subcutaneous administration, intradermal administration, topical administration, intranasal administration, intrapulmonary administration, and rectal administration, but is not limited thereto.

Herein, the subject to which the pharmaceutical composition may be administered may be a mammal, and specifically, a mouse, a rat, a guinea pig, a hamster, a rabbit, a dog, a cat, a horse, a cow, a goat, a monkey, a pig, a chicken, an ape, or a human.

A suitable dose of the pharmaceutical composition of the present disclosure may be variously prescribed by factors, such as a formulation method, an administration type, age, weight, and sex of a patient, a pathological condition, food, an administration time, an administration route, an excretion rate, and response susceptibility. The dose of the pharmaceutical composition according to the present disclosure may be administered once to several times in a dose of 0.001 mg/kg to 100 mg/kg based on an adult. The dose should not be construed as limiting the scope of the present disclosure in any aspect.

The pharmaceutical composition may include a pharmaceutically acceptable carrier. Here, the “pharmaceutically acceptable” means that the composition does not inhibit the activity of the active ingredient and does not have more than adaptable toxicity of an application (prescription) target. The carrier may be included in an amount of about 1 wt % to about 99.99 wt %, preferably about 90 wt % to about 99.99 wt %, based on the total weight of the pharmaceutical composition of the present disclosure.

When the pharmaceutical composition is prepared in a parenteral formulation, the pharmaceutical composition may be formulated in the form of injections, transdermal agents, nasal inhalation, and suppositories together with a suitable carrier according to methods known in the art. When formulated in injections, the suitable carrier may include sterile water, ethanol, polyols such as glycerol or propylene glycol, or mixtures thereof, preferably Ringer's solution, phosphate buffered saline (PBS) containing triethanol amine or sterile injectable solution, isotonic solutions such as 5% dextrose, or the like. The suitable carrier is known in the art regarding the formulation of pharmaceutical composition.

The pharmaceutical composition of the present disclosure may additionally include any compound or natural extract known to have a preventive or treatment effect on liver disease or may be used in combination with a separate composition (hereinafter referred to as a therapeutic agent) containing the substance, in addition to the active ingredient.

The “combination” includes two or more therapeutic agents in the same or separate formulations. When the pharmaceutical composition is prepared in a separate formulation from the therapeutic agent and used in combination, the pharmaceutical composition and the therapeutic agent may be mixed and administered “simultaneously” or may also be administered sequentially. When the pharmaceutical composition is administered sequentially with another therapeutic agent, the pharmaceutical composition may be administered before and/or after the administration of the therapeutic agent.

Yet another aspect of the present disclosure provides a use of a TSG-6 fragment or a pharmaceutical composition including the TSG-6 fragment as an active ingredient for preventing or treating liver disease.

Yet another aspect of the present disclosure provides a method for preventing or treating liver disease, including administering a TSG-6 fragment or a pharmaceutical composition including the TSG-6 fragment as an active ingredient to a subject. At this time, the TSG-6 fragment, the pharmaceutical composition, administration, prevention, and treatment are the same as described above.

The “subject” may be a mammal, preferably a human. In addition, the subject may be a patient suffering from liver disease or a subject likely to suffer from liver disease.

The administration route, dose, and number of administration times of the TSG-6 fragment or the pharmaceutical composition may be administered to the subject in various methods and amounts depending on a patient's condition and the presence or absence of side effects, and the optimal administration method, dose, and number of administration times may be selected within an appropriate range by those skilled in the art. In addition, the TSG-6 fragment or the pharmaceutical composition may be administered in combination with any compound or natural extract known to have a treatment effect on liver disease, or may be formulated in the form of a combined preparation with other drugs.

Hereinafter, preferred Examples are presented to assist understanding of the present disclosure. However, the following Examples are just provided to more easily understand the present disclosure, and the contents of the present disclosure are not limited by the following Examples.

### Example 1. Design of Novel Peptide Candidates Binding to CD44 and MMP-14 and Confirmation of Binding Affinity

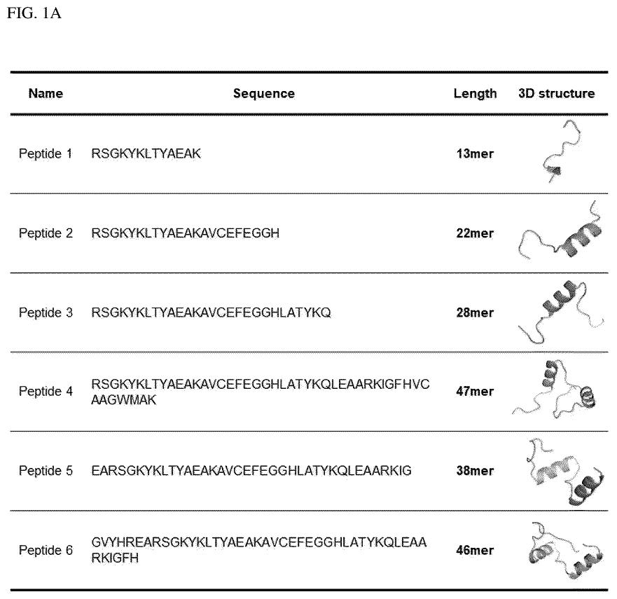

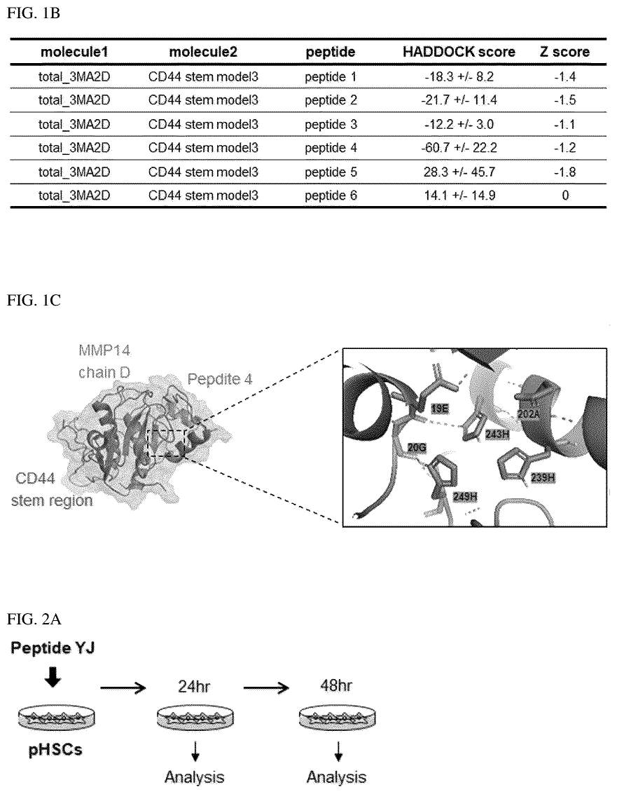

A sequence that mimicked a binding site of TSG-6 interacting with CD44 and MMP-14 was predicted, and candidate peptide sequences similar to the binding site were structured using the AI-based protein structure prediction program AlphaFold (FIG. 1A).

Specifically, the amino acid sequence of each peptide was input into the AlphaFold version 2.1.0 program, and the AlphaFold program was run with max_template_data=2020 May 14 as a parameter. Thereafter, an amino acid 3D structure file in a pdb format was obtained from AlphaFold, and then used in the protein docking program.

The binding affinity of the candidate peptides (6 types), predicted as above, to CD44 and MMP-14 was analyzed using HADDOCK2.4, an online-based protein docking program (FIG. 1B). In addition, a binding structure of peptide 4, which had the best binding affinity among the peptides, with MMP-14 and CD44 was predicted and illustrated in FIG. 1C.

As a result, when the binding structure of peptide 4 with MMP14 and CD44 was analyzed, it was confirmed that peptide 4 directly bound to enzyme active sites 202A, 239H, 243H, and 249H of MMP14. Through the results, it was predicted that peptide 4 may inhibit the CD44 cleavage activity of MMP14.

In the present disclosure, the peptide 4 was named “peptide YJ”.

### Example 2. Preparation of Peptide YJ

The peptide YJ according to the present disclosure was prepared by request to Peptron Inc.

### Example 3. Confirmation of Liver Fibrosis Inhibition Effect of Peptide YJ on Human pHSCs

A liver fibrosis inhibition effect of peptide YJ on human pHSCs was confirmed through analysis of cell survival and expression of cell activation markers.

Specifically, the cell survival was confirmed by the following method.

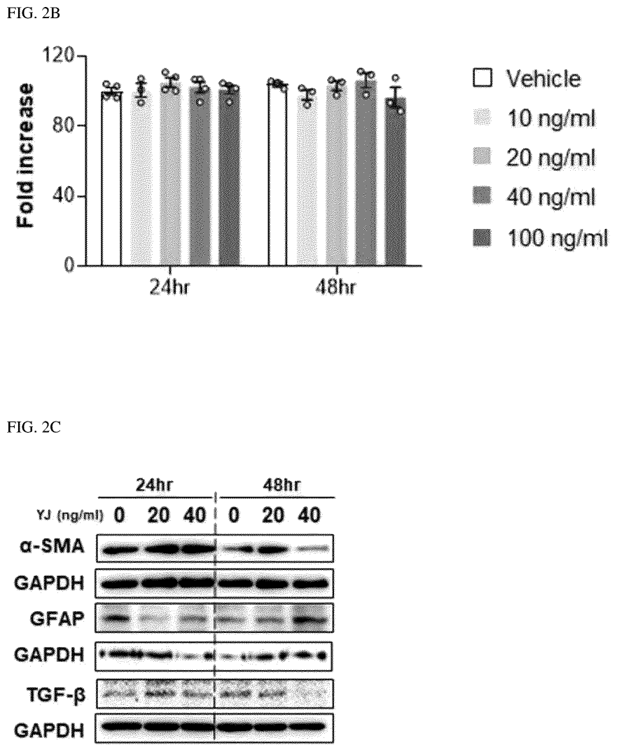

100 μl of human pHSCs (5×10⁴ cells/ml) were seeded into 96 wells and cultured overnight. The cells were treated with peptide YJ at a concentration of 0, 20, 40, or 100 ng/ml and then further cultured. After 24 and 48 hours, cell survival for each cell was confirmed through MTS assay (Cell titer proliferation assay, G3580, Promega) (FIG. 2A). At this time, human pHSCs treated with PBS were used as a control group (vehicle).

As a result, as illustrated in FIG. 2B, no changes in cell survival and proliferation were observed by peptide YJ treatment compared to the control group.

To confirm the expression of cell activation markers, human pHSCs (5× 10⁵ cells/ml) were treated with peptide YJ at a concentration of 20 or 40 ng/ml, and then proteins were extracted and subjected to Western blotting. At this time, as primary antibodies, anti-α-SMA antibody (Sigma-Aldrich, A5228), anti-GAPDH antibody (Bio-rad, MCA4739), anti-GFAP antibody (Dako, z0334), and anti-TFG-β antibody (Cell Signaling Technology, 3711) were used. In addition, as secondary antibodies, Goat anti-mouse IgG F(ab′) 2 antibody (HRP conjugate) (Enzo, ADI-SAB-100-J) and Goat anti-rabbit IgG antibody (HRP conjugate) (Enzo, ADI-SAB-300-J) were used.

As a result, as illustrated in FIG. 2C, it was confirmed that the expression of activation markers of pHSCs decreased in the 40 ng/ml peptide YJ treated group compared to the control group, while the expression of GFAP (glial fibrillary acidic protein) as an inactivation marker increased.

Through the results, it was confirmed that peptide YJ may be used as a therapeutic agent for liver fibrosis by inhibiting the activation of pHSCs without affecting the survival of pHSCs.

### Example 4. Confirmation of CD44 Cleavage Inhibition Effect by Peptide YJ Treatment in Human pHSCs

In order to confirm the mechanism explaining HSC inactivation caused by peptide YJ of Example 3, inhibitory ability of peptide YJ on the CD44 cleavage was confirmed in pHSCs.

Specifically, pHSCs were treated with peptide YJ (40 ng/ml) for 0, 6, 12, or 24 hours, and then nuclear proteins were isolated. Western blotting was performed on the isolated nuclear proteins to confirm the expression of CD44 and CD44ICD. The nuclear proteins were obtained by a nuclear protein fractionation method.

At this time, the primary antibodies were used with anti-CD44 antibody (Abcam, ab157107), anti-CD44ICD antibody (Cosmo Bio, KAL-KO601), and anti-LAMIN B1 antibody (Abcam, ab16048), and the secondary antibody was used with Goat anti-Rabbit IgG antibody (HRP conjugate) (Enzo, ADI-SAB-300-J). In addition, human pHSCs treated with PBS were used as a control group. The CD44 and CD44ICD expression results were normalized to the expression level of LAMIN B1, which was an internal control group, and expressed as mean±SEM values obtained from the same experiment repeated three times.

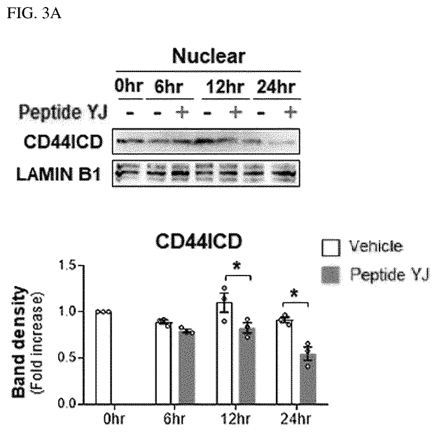

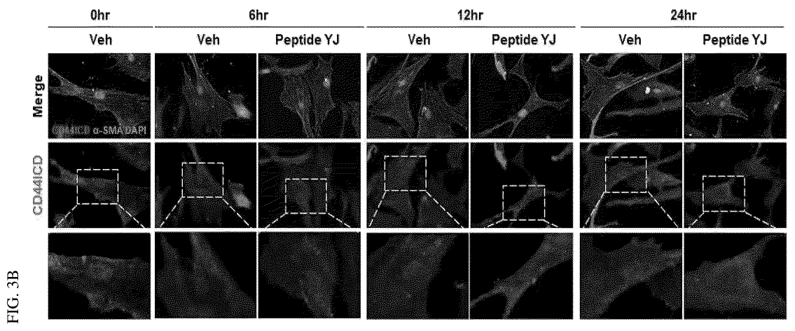

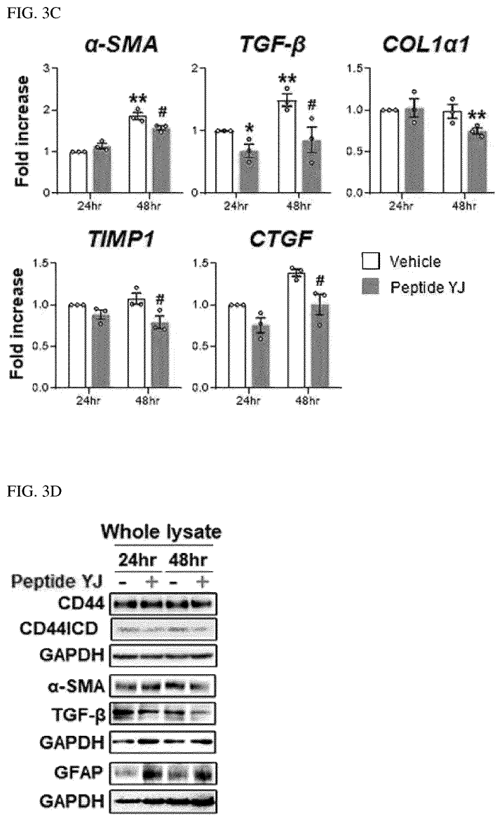

As a result, as illustrated in FIG. 3A, when peptide YJ (40 ng/ml) was treated for 24 hours compared to the control group, the nuclear expression and accumulation of CD44ICD of pHSCs were significantly reduced.

The cleavage of CD44 and the expression of the activation marker of pHSCs by peptide YJ treatment were confirmed through immunofluorescence. At this time, human pHSCs treated with PBS were used as a control group.

Specifically, human pHSCs (5×10⁴ cells/ml) were seeded on coverslips and then cultured overnight. The cells were treated with peptide YJ at a concentration of 40 ng/ml and further cultured for 6, 12, and 24 hours, respectively. The cells were fixed and permeabilized by treating each cell with 4% paraformaldehyde and 0.01% Triton X-100. To inhibit non-specific binding of antibodies, blocking (Dako, X9090) was performed, and then the primary antibodies were treated and reacted overnight at 4° C. At this time, the primary antibodies used were anti-CD44ICD antibody (Cosmo Bio, KAL-KO601) and α-SMA (Sigma-Aldrich, A5228). Thereafter, the cells were washed with TBS, treated with a secondary antibody, and reacted at room temperature for 30 minutes. The secondary antibody used were Alexa Fluor 488-conjugated chicken anti-mouse IgG antibody (Invitrogen, A21200) or Alexa Fluor 568-conjugated goat anti-rabbit IgG antibody (Invitrogen, A10042). The cells were washed with TBS, and then treated with 4′,6-diamidino-2-phenylinole (DAPI, VECTASHIELD, H-1200-10) to perform nuclear staining. Finally, the coverslip was fixed on a slide glass using a mounting solution and then observed with a confocal microscope DMi8-S (Leica Microsystems) or CLSM21 (Carl Zeiss Inc.).

As a result, as illustrated in FIG. 3B, the expression of CD44ICD was observed in the nucleus of the control group. On the other hand, in the case of the peptide YJ treated group, it was confirmed that the expression of CD44ICD in the nucleus decreased 12 hours after peptide YJ treatment, and no expression of CD44ICD in the nucleus was observed 24 hours after peptide YJ treatment. The expression of α-SMA, a HSC activation marker, was also clearly observed in the control group, whereas in the peptide YJ treated group, the expression of α-SMA was confirmed to be decreased after 12 hours of peptide treatment. In addition, it was confirmed that the expression of α-SMA in the nucleus was significantly decreased after 24 hours of peptide treatment.

In addition, after pHSCs were treated with peptide YJ for 24 and 48 hours by the same method as above, respectively, RNA and protein were extracted (whole lysate) and the expression of pHSCs activation markers was confirmed through RT-qPCR (FIG. 3C) and Western blot (FIG. 3D). Western blot was performed using the same method as in Example 2, and RNA extraction and RT-qPCR were performed as follows. At this time, human pHSCs treated with PBS were used as a control group.

First, total RNA was extracted from each cell using a Trizol solution (Invitrogen, 15596-026), and then the concentration and purity of RNA were confirmed using a nanodrop. Thereafter, complementary DNA (cDNA) was synthesized using the RNA as a template using the SuperScript First-strand Synthesis System (Invitrogen, 18064014). The cDNA synthesized by the method was used as sample DNA in quantitative real-time PCR (qPCR).

The quantitative real-time PCR (qPCR) was performed using Power SYBR Green Master Mix (Applied Biosystem), and the results were analyzed by a AACt method. The qPCR results were analyzed for significance using Mean±SEM values of three independent experiments through one-way ANOVA followed by post hoc Tukey's test.

As a result, as illustrated in FIGS. 3C and 3D, it was confirmed that the expression of fibrosis markers α-SMA (α-smooth muscle actin), TGF-β (transforming growth factor-β), COL1α1 (collagen type 1α1 chain), TIMP1 (metallopeptidase inhibitor 1), and CTGF (connective tissue growth factor) decreased in the peptide YJ treated group compared to the control group. In addition, it was confirmed that GFAP, an inactivation marker of pHSCs, increased in the peptide YJ treated group compared to the control group.

Through the results, it was confirmed that peptide YJ may inhibit the activation of pHSCs by inhibiting the cleavage of CD44 into CD44ICD and reducing the nuclear accumulation of CD44ICD.

### Example 5. Confirmation of Treatment Effect of Peptide YJ on Alcohol-Related Liver Disease Using an Alcohol-Related Liver Disease Mouse Model

### Example 5.1. Establishment of Alcohol-Related Liver Disease Mouse Model

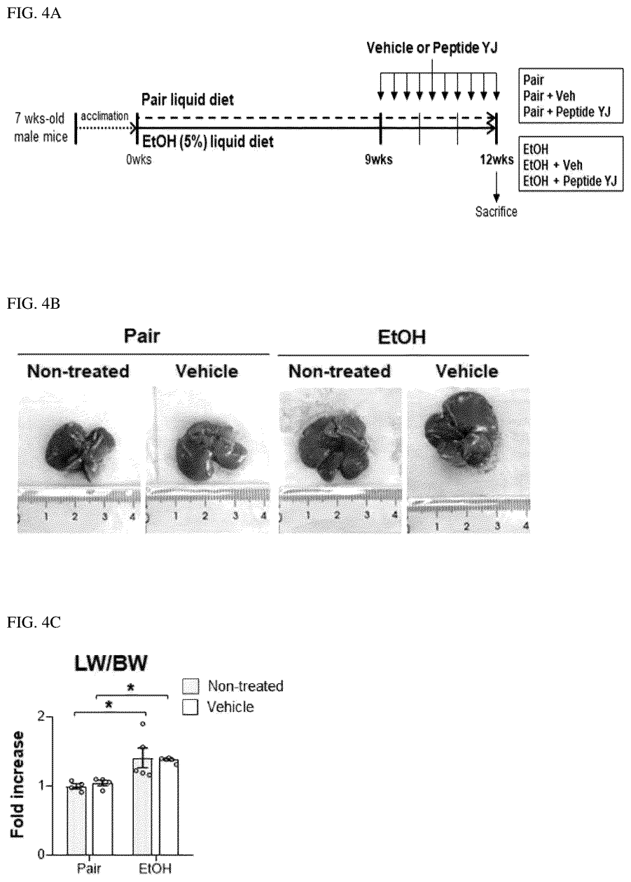

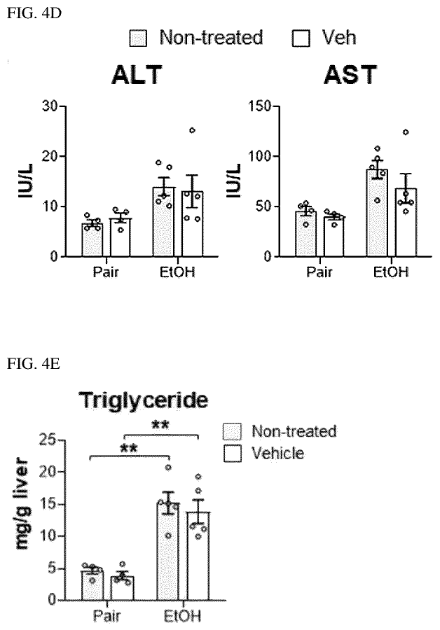

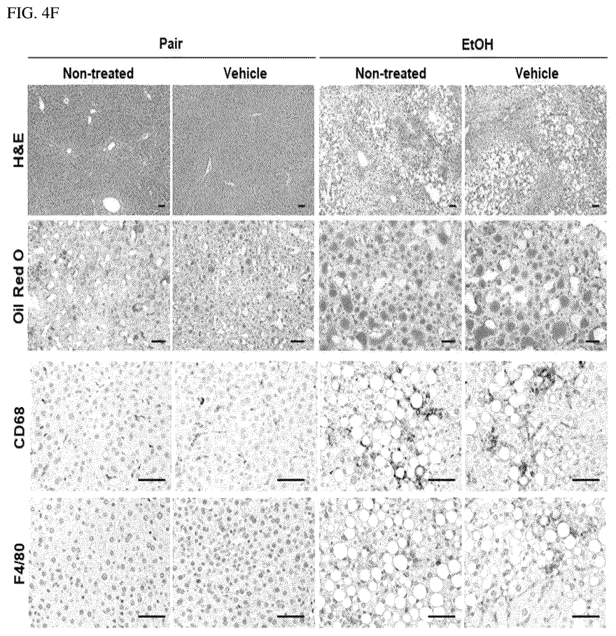

To prepare alcohol-related liver disease mice, 8-week-old C57BL/6 male mice were fed with a Lieber DeCarli liquid diet (Dyets, 710260) containing 5% EtOH for 9 weeks, and then injected intraperitoneally with a vehicle (PBS) every other day along with feeding liquid diet for additional 3 weeks. As a control group for the EtOH diet, mice fed with a pair Lieber DeCarli liquid diet (Dyets, 710027) having the same total calories as the EtOH diet were used (FIG. 4A).

### Example 5.2. Phenotypic Analysis of Alcohol-Related Liver Disease Mouse Model

Blood and the liver tissue were collected from alcohol-related liver disease mice prepared by the method of Example 5.1 to confirm the liver disease pathology of the mice.

### Example 5.2.1. Measurement of Liver Tissue Sizes and Liver Damage Markers

The shape and weight of the liver tissue obtained by the method of Example 5.2 were confirmed. As a result, it was confirmed that the liver size of the mice that consumed the EtOH diet was enlarged compared to a control group (pair). In addition, while the liver of the control group was reddish brown, the liver of the EtOH diet group was yellow and pale (FIGS. 4B and 4C).

In addition, the levels of serum aminotransferase (ALT) and aspartate aminotransferase (AST), which were liver damage markers, were measured by the following method.

Specifically, 20 μl of mouse serum was mixed with 100 μl of a GOT buffer or GPT buffer, and then reacted at 37° C. Thereafter, the mixture was added with a 0.0198% 2,4-dinitrophenylhydrazine colorimetric solution (100 μl), and then further reacted at room temperature for 20 minutes. Thereafter, the mixture was added with 0.4 N NaOH (1 ml) to terminate the reaction, and absorbance (505 nm) was measured. The absorbance was expressed in international units per liter (IU/L).

As a result, as illustrated in FIG. 4D, the serum AST and ALT levels of the EtOH diet group tended to increase compared to the control group (Pair).

### Example 5.2.2. Confirmation of Triglyceride Accumulation in Liver Tissue

Triglycerides in the liver tissue obtained by the method of Example 5.2 were measured using the following method.

In addition, the accumulation of triglycerides in the liver tissue was confirmed using Oil red O staining.

Specifically, the liver tissue extracted from the mice was embedded in OCT and frozen-stored at −80° C. The liver tissue embedded in OCT was cut into 6 μm-thick sections to produce tissue section slides and then fixed by treating 4% paraformaldehyde. The tissue section slides were washed with distilled water and treated with 100% propylene glycol (PEG, Sigma-Aldrich), and triglycerides were stained with Oil Red O (Sigma-Aldrich). After staining, the section slides were washed with 60% PEG, and the nuclei were counterstained with hematoxylin. The liver tissue section slides produced as described above were observed under a microscope to confirm the accumulation of triglycerides in the liver tissue.

As a result, as illustrated in FIG. 4E, it was confirmed that the triglycerides (TG) in the liver tissue of the EtOH diet group significantly increased compared to the control group (pair).

In addition, it was confirmed that larger lipid droplet were accumulated in the liver tissue of the EtOH diet group than in the control group (FIG. 4F).

### Example 5.2.3. Confirmation of Inflammatory Response in Liver Tissue

Immunohistochemical staining was performed on the liver tissue obtained by the method of Example 5.2 using anti-CD68 antibody (Abcam, ab12521) and anti-F4/80 antibody (Abcam, ab6640).

Specifically, the liver tissue extracted from the mouse was fixed by treating 10% neutral buffered formalin, and then embedded using paraffin. The liver tissue paraffin block was cut into 4 μm-thick sections to produce tissue section slides. After deparaffinization and hydration processes of the tissue section slides, 3% hydrogen peroxide was treated to block endogenous peroxidase. Then, the section slides were treated with 10 mM sodium citrate buffer (pH 6.0) and heated for 10 minutes or treated with 0.2% pepsin to induce antigen retrieval. To inhibit non-specific binding of antibodies, blocking (Dako, X9090) was performed, and then the primary antibodies were treated and reacted overnight at 4° C. At this time, the primary antibodies used were anti-CD68 antibody (Abcam, ab125212) and anti-F4/80 antibody (Abcam, ab6640). The section slides were washed with TBST and then treated with secondary antibodies and reacted. The secondary antibodies used were EnVision+ Single Reagents (HRP, Rabbit) (Dako, K4003) and Goat anti-rat IgG-heavy and light chain antibodies (HRP conjugated) (BETHYL, A110-105P). Thereafter, the section slides were washed with TBST and treated with 3,3′-diaminobenzidine (DAB) (Dako, K3466) to develop the color. After the color development, the tissues of the section slides were dehydrated, mounted, fixed, and then observed under a microscope.

As a result, it was confirmed that the expression of macrophage markers CD68 and F4/80 increased. Through the results, it was confirmed that the inflammatory response in the liver tissue increased by the EtOH diet (FIG. 4F).

### Example 5.2.4. Confirmation of Collagen Accumulation in Liver Tissue

In most chronic liver diseases, severe inflammatory responses were accompanied by fibrosis. Accordingly, the degree of fibrosis in the liver tissue of the alcohol-related liver disease mouse model established by the method of Example 5.1 in the present disclosure was confirmed through the expression of fibrosis markers using RT-qPCR and Western blot. In addition, the accumulation of collagen in the liver tissue and the expression of α-SMA were confirmed. In addition, the concentration of collagen in the liver tissue was confirmed through Sirius red staining, and the expression of α-SMA was confirmed through immunohistochemical staining. At this time, RT-qPCR and Western blot were performed in the same manner as in Example 2, and immunohistochemical staining (IHC) was performed in the same manner as in Example 5.2.3. In IHC, an anti-α-SMA antibody was used as the primary antibody.

Sirius red staining was performed by the following method.

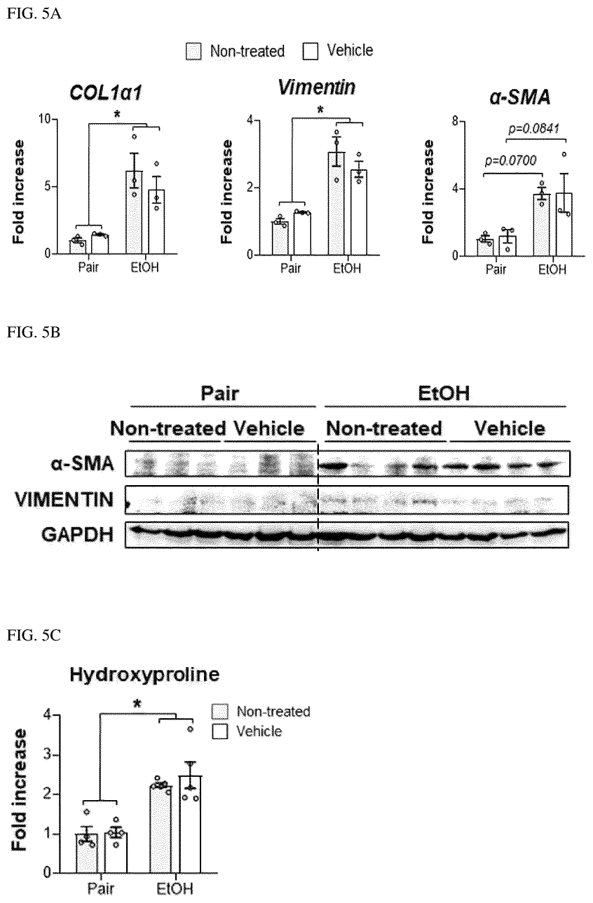

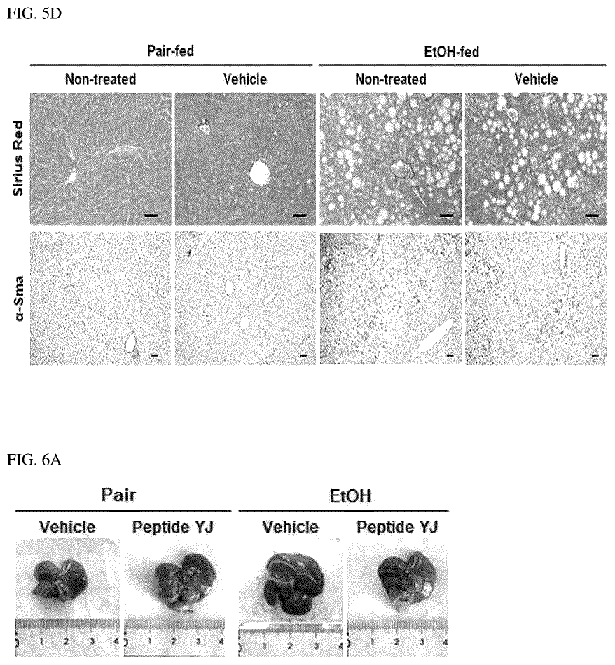

After deparaffinization and hydration of the liver tissue section slides prepared in the same manner as in Example 5.2.3, the section slides were treated with 0.1% Sirius red F3B (Sigma-Aldrich) and 0.1% Fast Green (Sigma-Aldrich) solutions containing saturated picric acid (Sigma-Aldrich) and reacted for 20 minutes. After the staining reaction was completed, the section slides were dehydrated, mounted, and observed under a microscope. As a result, as illustrated in FIGS. 5A and 5B, it was confirmed that compared to the control group (pair), in the EtOH diet group, the expression of mRNA of COL1α1 was increased and the expression of mRNA and protein of α-SMA and vimentin was increased. In addition, it was confirmed that the accumulated collagen in the liver tissue also significantly increased in the EtOH diet group (FIGS. 5C and 5D). In addition, it was confirmed that the expression of α-SMA in the liver tissue in the EtOH diet group increased compared to the control group (pair) (FIG. 5D).

Through the results, it was confirmed that the alcohol-related liver disease mouse model was normally established through the method of Example 5.1, and the mouse model established by the method was used to evaluate the liver disease therapeutic activity of peptide YJ.

In addition, through the results, since there was no difference in disease phenotype between mice that were not administered with any substance (non-treated) and mice administered with a vehicle (PBS), in subsequent analyses, the mice administered with the vehicle (PBS) in the EtOH diet group were used as a control group.

### Example 5.3. Confirmation of Liver Damage Treatment Effect of Peptide YJ in Alcohol-Related Liver Disease Mouse Model

The alcohol-related liver disease mouse model established by the method of Example 5.1 was administered vehicle (PBS) or peptide YJ (3.3 μg/kg) intraperitoneally every other day for three weeks, and then blood was collected and liver tissue was extracted to confirm the liver disease treatment effect of peptide YJ.

### Example 5.3.1. Measurement of Liver Tissue Sizes and Tissue Damage Markers

The weight and degree of tissue damage of the liver tissue extracted from the experimental mice of Example 5.3 were measured in the same manner as in Example 5.2.1.

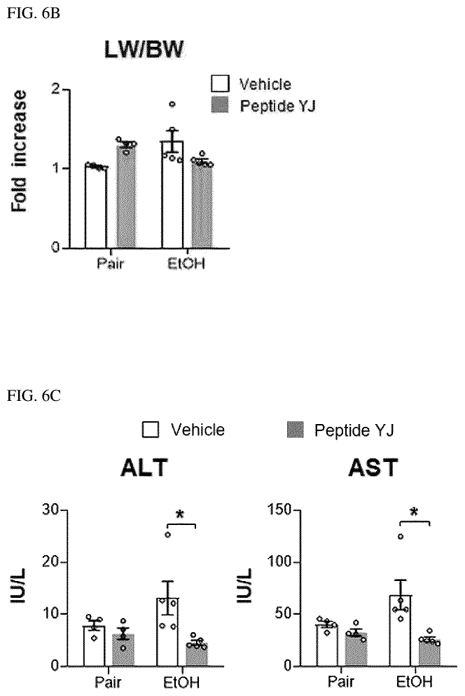

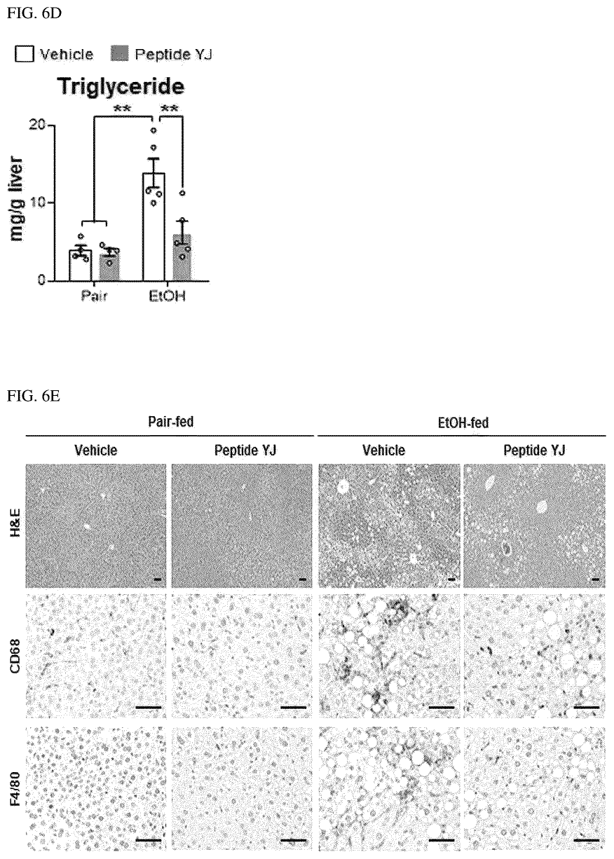

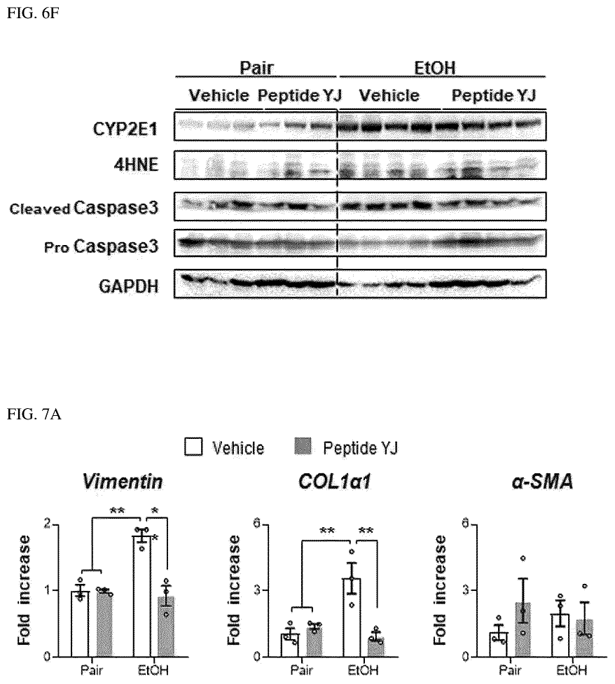

As a result, as illustrated in FIGS. 6A and 6B, the size and weight of the liver tissue did not show statistically significant differences between the groups. However, in the EtOH diet group, the AST and ALT levels decreased in the peptide YJ administered group compared to the vehicle administered group (FIG. 6C).

### Example 5.3.2. Confirmation of Triglyceride Accumulation in Liver Tissue

The degree of triglyceride accumulation in the liver tissue extracted from the experimental mice of Example 5.3 was confirmed in the same manner as in Example 5.2.2.

As a result, as illustrated in FIG. 6D, it was confirmed that the amount of triglycerides in the liver tissue of the peptide YJ administered group was significantly reduced compared to the vehicle administered group in the EtOH diet group. In addition, the H&E staining results also confirmed that the size and number of lipid droplet in the liver tissue were significantly reduced (FIG. 6E).

### Example 5.3.3. Confirmation of Inflammatory Response in Liver Tissue

The degree of inflammatory response in the liver tissue extracted from the experimental mice of Example 5.3 was confirmed in the same manner as in Example 5.3.2.

As a result, as illustrated in FIG. 6E, it was confirmed that in the EtOH diet group, the expression of CD68 and F/80 was significantly reduced in the peptide YJ administered group compared to the vehicle administered group.

In addition, the expression of alcohol metabolism markers and apoptosis marker proteins was confirmed through Western blot. Western blot was performed in the same manner as in Example 2. At this time, the primary antibodies used were anti-CYP2E1 antibody (Merck, ab125212), anti-4HNE antibody (Abcam, ab46545), anti-cleaved caspase-3 antibody (Cell Signaling Technology, 9661s), anti-caspase-3 antibody (Cell Signaling Technology, 9662s), and anti-GADPH antibody.

As a result, as illustrated in FIG. 6F, the expression of CYP2E1 and 4HNE in the vehicle administered group and the peptide YJ administered group in the EtOH diet group did not show a significant difference. However, it was confirmed that the expression of cleaved caspase-3, an apoptosis marker, decreased in the peptide YJ administered group compared to the vehicle administered group.

### Example 5.3.4. Confirmation of Collagen Accumulation in Liver Tissue

The degree of fibrosis in the liver tissue extracted from the experimental mice of Example 5.3 was confirmed in the same manner as in Example 5.2.4.

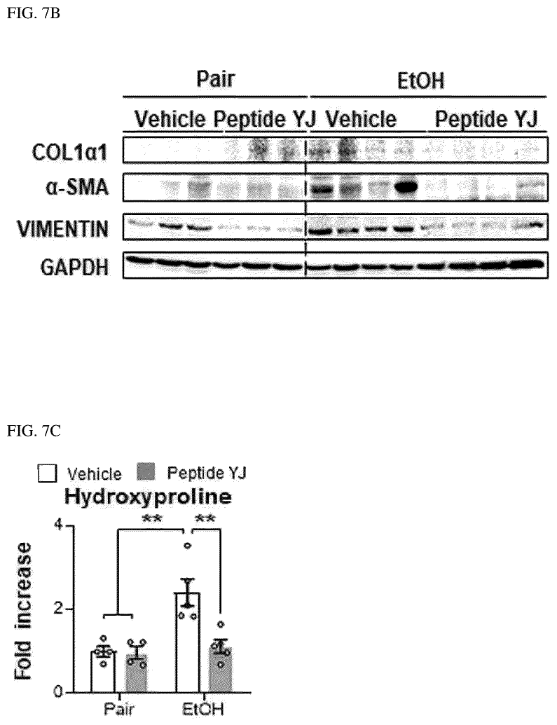

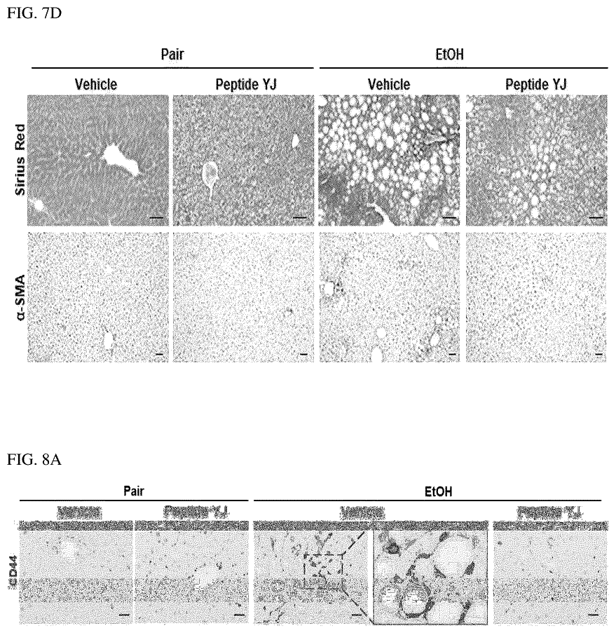

As a result, as illustrated in FIGS. 7A and 7B, it was confirmed that in the EtOH diet group, the mRNA and protein expression of liver fibrosis markers (α-SMA, vimentin, and collagen1α1) in the liver tissue of peptide YJ administered group was significantly reduced compared to the vehicle administered group. In addition, even in a result of the hydroxyproline assay, Sirius red staining, and IHC (α-SMA) of the EtOH diet group, it was confirmed that collagen accumulation in the liver tissue in the peptide YJ administered group was significantly reduced compared to the vehicle administered group (FIGS. 7C and 7D).

Through the results, it was confirmed that peptide YJ may alleviate liver fibrosis, a pathological condition of alcohol-related liver disease.

### Example 6. Confirmation of CD44 Cleavage Inhibition Effect of Peptide YJ in Alcohol-Related Liver Disease Mouse Model

The expression of CD44 and CD44ICD in liver tissues extracted from the experimental mice of Example 5.3, or nuclear proteins or whole proteins (whole lysate) of liver tissues, was confirmed through immunohistochemical staining and Western blot. The immunohistochemical staining was performed using anti-CD44 antibody and anti-CD44ICD antibody by the method of Example 5.2.3, and Western blot was performed by the method of Examples 2 and 4.

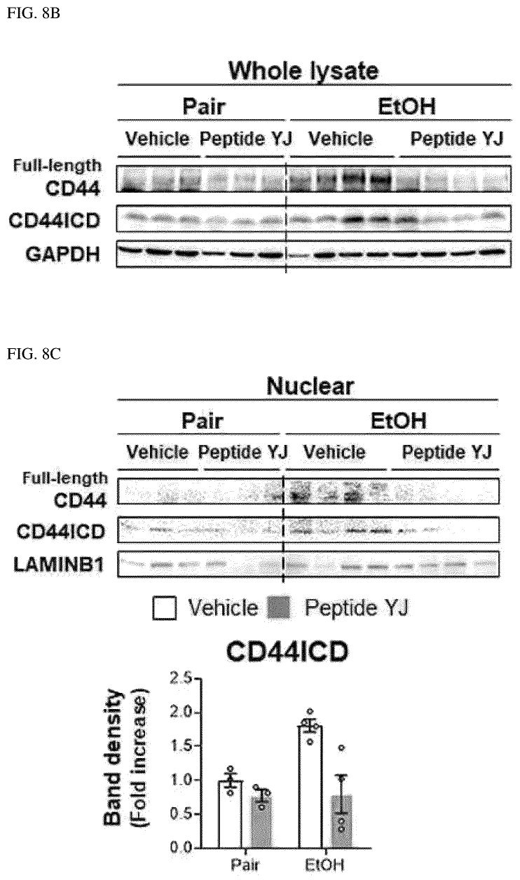

As illustrated in FIG. 8A, it was confirmed that the expression of CD44 increased by the EtOH diet, and the expression of CD44 decreased by the administration of peptide YJ.

In addition, even in the Western blot result of the whole lysate, it was confirmed that the expression of CD44 and CD44ICD was reduced in the peptide YJ administered group compared to the vehicle administered group in the EtOH diet group (FIG. 8B). In particular, in the nuclear protein expression result, it was confirmed that the expression of CD44ICD was significantly reduced in the peptide YJ administered group compared to the vehicle administered group in the EtOH diet group (FIG. 8C).

Through the result, it was confirmed that peptide YJ may inhibit liver fibrosis by inhibiting the cleavage of CD44.

As a TSG-6 mimetic, peptide YJ well mimicked the mechanism of action of TSG-6 and showed the same therapeutic effect against liver disease. Therefore, the peptide YJ may be used as a pharmaceutical composition for the treatment of liver diseases.

1. A tumor necrosis factor-inducible gene 6 protein (TSG-6) fragment.

2. The TSG-6 fragment of claim 1, wherein the TSG-6 fragment is a polypeptide represented by the following Structural Formula I:
[Core domain]−[C-terminal extension domain] (I)
In Structural Formula I,
the core domain is a polypeptide including an amino acid sequence represented by SEQ ID NO: 1, and
the C-terminal extension domain may have 0 to 33 amino acids sequentially deleted from the C-terminal to the N-terminal starting from an amino acid at position 33 of SEQ ID NO: 9.

3. The TSG-6 fragment of claim 2, wherein the C-terminal extension domain includes any one amino acid sequence selected from the group consisting of SEQ ID NOs: 7 to 9.

4. The TSG-6 fragment of claim 2, wherein the TSG-6 fragment consists of any one amino acid sequence selected from the group consisting of SEQ ID NOs: 1 to 4.

5. The TSG-6 fragment of claim 2, wherein the TSG-6 fragment consists of an amino acid sequence having 90% or more homology with the amino acid sequence represented by SEQ ID NO: 4.

6. The TSG-6 fragment of claim 1, wherein the TSG-6 fragment inhibits cleavage of CD44.

7. A polynucleotide encoding the TSG-6 fragment of claim 1.

8. A method for treating liver disease, comprising administering the TSG-6 fragment of claim 1 or a pharmaceutical composition comprising the TSG-6 fragment to a subject in need thereof.

9. The method for treating liver disease of claim 8, wherein the liver disease is selected from the group consisting of fatty liver, hepatitis, liver fibrosis, liver cirrhosis, liver failure, and liver cancer.

10. The method for treating liver disease of claim 8, wherein the liver disease is alcohol-related liver disease.


SEQUENCE LISTING (10 sequences)

SEQ ID NO: 1 - protein, 13 aa, synthetic construct
  source 1..13: note = core domain; note = peptide 1
  REGION 1..13
RSGKYKLTYAEAK

SEQ ID NO: 2 - protein, 22 aa, synthetic construct
  source 1..22: note = peptide 2
RSGKYKLTYAEAKAVCEFEGGH

SEQ ID NO: 3 - protein, 28 aa, synthetic construct
  source 1..28: note = Peptide 3
RSGKYKLTYAEAKAVCEFEGGHLATYKQ

SEQ ID NO: 4 - protein, 47 aa, synthetic construct
  source 1..47: note = peptide 4
RSGKYKLTYAEAKAVCEFEGGHLATYKQLEAARKIGFHVCAAGWMAK

SEQ ID NO: 5 - protein, 38 aa, synthetic construct
  source 1..38: note = peptide 5
EARSGKYKLTYAEAKAVCEFEGGHLATYKQLEAARKIG

SEQ ID NO: 6 - protein, 45 aa, synthetic construct
  source 1..45: note = peptide6
GVYHREARSGKYKLTYAEAKAVCEFEGGHLATYKQLEAARKIGFH

SEQ ID NO: 7 - protein, 8 aa, synthetic construct
  source 1..8: note = extension sequence of C
VCEFEGGH

SEQ ID NO: 8 - protein, 14 aa, synthetic construct
  source 1..14: note = extension sequence of C
VCEFEGGHLATYKQ

SEQ ID NO: 9 - protein, 33 aa, synthetic construct
  source 1..33: note = extension sequence of C
VCEFEGGHLATYKQLEAARKIGFHVCAAGWMAK

SEQ ID NO: 10 - protein, 277 aa, synthetic construct
  source 1..277: note = TSG-6; note = human
MIILIYLFLLLWEDTQGWGFKDGIFHNSIWLERAAGVYHREARSGKYKLTYAEAKAVCEFEGGHLATYKQLEAARKIGFHVCAAGWMAKGRVGYPIVKPGPNCGFGKTGIIDYGIRLNRSERWDAYCYNPHAKECGGVFTDPKQIFKSPGFPNEYEDNQICYWHIRLKYGQRIHLSFLDFDLEDDPGCLADYVEIYDSYDDVHGFVGRYCGDELPDDIISTGNVMTLKFLSDASVTAGGFQIKYVAMDPVSKSSQGKNTSTTSTGNKNFLAGRFSHL